(19) United States
(12) Patent Application Publication
(10) Pub. No.: US 2010/0203084 A1
(43) Pub. Date: Aug. 12, 2010

(54) METHOD FOR TREATING PAIN OR OPIOID DEPENDENCE USING A SPECIFIC TYPE OF NON-OPIOID AGENT IN COMBINATION WITH A SELECTIVE EXCITATORY-OPIOID-RECEPTOR INACTIVATOR

(76) Inventors: Stanley M. Crain, State College, PA (US); Ke-Fei Shen, Flushing, NY (US)

Correspondence Address: AMSTER, ROTHSTEIN & EBENSTEIN LLP, 90 PARK AVENUE, NEW YORK, NY, 10016, US

(21) Appl. No.: 12/312,164

(22) PCT Filed: Oct. 12, 2007
(86) PCT No.: PCT/US2007/021836

§ 371 Date: Apr. 12, 2010

Related U.S. Application Data
(60) Provisional application No. 60/855,775, filed on Nov. 1, 2006.

(51) Int. Cl. A61K 39/106; A61K 31/195; A61K 31/40; A61K 31/522; A61K 31/352; A61K 31/485; A61K 31/215; A61K 31/35; A61K 33/06; A61K 33/04; A61P 29/00

(52) U.S. Cl. 424/236.1; 514/561; 514/424; 514/263.34; 514/455; 514/282; 514/279; 514/529; 514/459; 424/682; 424/709

(57) ABSTRACT

The present invention provides a method for treating pain in a subject in need of treatment, by administering to the subject a non-opioid agent in combination with a selective excitatory-opioid-receptor inactivator, in amounts effective to treat pain in the subject. Also disclosed is a method for treating opioid-withdrawal effects in a subject in need of treatment, by the administration to the subject of a non-opioid agent in combination with a selective excitatory-opioid-receptor inactivator, in amounts effective to treat opioid-withdrawal effects in the subject. Finally, the present invention provides a pharmaceutical composition comprising a non-opioid agent and a selective excitatory-opioid-receptor inactivator, and a pharmaceutically-acceptable carrier.

### BACKGROUND OF THE INVENTION

Morphine and many other opioid agonists have analgesic effects that are mediated by their activation of inhibitory opioid receptors on nociceptive (pain-mediating) neurons (24). Accordingly, these opioids are administered to relieve severe pain. Morphine and many other opioid agonists, however, also have been shown to activate excitatory opioid receptors on nociceptive neurons, thereby attenuating the analgesic potency of the opioid agonists, and resulting in the development of anti-analgesia, hyperexcitability, hyperalgesia, physical dependence, psychological dependence, addiction, tolerance, and other adverse excitatory effects (25, 28, 39).

Previous patents have disclosed that the analgesic potency of bimodally-acting opioid agonists can be enhanced, and the tolerance/dependence liability reduced, by co-administering the bimodally-acting opioid agonists with ultra-low doses of selective excitatory-opioid-receptor antagonists (e.g., U.S. Pat. Nos. 5,472,943; Re 36,547; 5,580,876; and 5,767,125). Excitatory-opioid-receptor antagonists are compounds that bind to and inactivate excitatory opioid receptors at low doses that are not effective in inactivating inhibitory opioid receptors on neurons in nociceptive (pain) pathways (25). Selective excitatory-opioid-receptor antagonists attenuate excitatory, but not inhibitory, opioid receptor functions in nociceptive pathways of the peripheral and central nervous systems. As a result, symptoms associated with activation of excitatory opioid receptors (e.g., anti-analgesia, hyperalgesia, hyperexcitability, physical dependence, psychological dependence, and tolerance effects) are blocked, while the analgesic effects of the bimodally-acting opioid agonists, which are mediated by the inhibitory opioid receptors, are enhanced (25, 28, 39).

Previous patents have further disclosed that ultra-low doses of naltrexone, alone or in combination with low-dose methadone (e.g., U.S. Pat. No. Re 36,547), and ultra-low doses of other excitatory-opioid-receptor antagonists alone (e.g., U.S. Pat. Nos. 5,580,876 and 5,767,125), can provide effective, long-term maintenance treatment for opioid addiction after acute detoxification, and can prevent relapse to drug abuse. Furthermore, preclinical studies have suggested that ultra-low doses of selective excitatory-opioid-receptor antagonists can be administered alone to chronic pain patients to enhance the analgesic potency and reduce the tolerance/dependence liability of endogenous opioid peptides, such as enkephalins, dynorphins, and endorphins, which are elevated in chronic pain patients (28).

A long-standing need has existed to develop a non-opioid or non-narcotic method for treating pain that does not produce the kinds of adverse excitatory effects associated with the administration of opioids. The present invention satisfies this need.

### SUMMARY OF THE INVENTION

The present invention provides a method for treating pain in a subject comprising administering to the subject a non-opioid agent in combination with a selective excitatory-opioid-receptor inactivator, in amounts effective to treat pain in the subject.

The present invention also provides a method for treating opioid-withdrawal effects in a subject comprising administering to the subject a non-opioid agent or non-narcotic in combination with a selective excitatory-opioid-receptor inactivator, in amounts effective to treat opioid-withdrawal effects in the subject.

The present invention further provides a pharmaceutical composition comprising a non-opioid agent and a selective excitatory-opioid-receptor inactivator, and a pharmaceutically-acceptable carrier.

Additional objects of the present invention will be apparent in view of the description which follows.

### BRIEF DESCRIPTION OF THE FIGURES

FIGS. 1A and 1B show that cotreatment of mice with ultra-low-dose naltrexone (NTX) or low-dose cholera toxin B subunit (CTX-B) blocks acute thermal hyperalgesic effects of 1 mg/kg N-methyl-D-aspartate (NMDA), thereby resulting in prominent, long-lasting analgesia. Presented are time-effect curves of hot-water-immersion (52° C.) tail-flick nociceptive tests (29). A: Administration of 1 mg/kg NMDA (s.c.) resulted in onset of decreases in tail-flick latencies (indicating hyperalgesia), which lasted for >5 h after drug injection (). Cotreatment (s.c.) with 1 mg/kg NMDA and either ultra-low-dose naltrexone (NTX) (0.1 ng/kg) or low-dose CTX-B (0.1 mg/kg) blocked NMDA-evoked hyperalgesia, and resulted in prominent analgesia (measured by increases in tail-flick latencies) that lasted for 5-6 h (◯ and ▾, respectively). B: Cotreatment of two other groups of mice with an initial injection of 1 mg/kg NMDA 0.1 ng/kg NTX, followed by a second injection of high-dose NTX (1 mg/kg) at 3 h after the initial injection, resulted in rapid attenuation of the initially prominent hyperalgesia (◯) and analgesia (▾). A control group injected with saline showed no significant alterations in baseline tail-flick latencies (). These results provide evidence that the observed hyperalgesia and analgesia may be mediated by opioid receptors activated by the NMDA-stimulated release of endogenous opioid agonists. Control assays with ultra-low-dose NTX or CTX-B alone showed no alterations in tail-flick latencies (data not shown). All drugs were injected subcutaneously. All mice used in these and subsequent experiments were male (Swiss-Webster), except for those in FIG. 2. In all figures, n=8 for each curve; error bars indicate S.E.M. A dashed line has been inserted in some figures to facilitate visualization of the hyperalgesic effects.

FIG. 2 illustrates that cotreatment of mice with ultra-low-dose NTX also blocks the hyperalgesic effects of 1 mg/kg glutamic acid, and results in prominent analgesia. Administration of 1 mg/kg dl-glutamic acid (s.c.) resulted in long-lasting hyperalgesia (>6 h)—an effect similar to that produced by NMDA (; cf. FIG. 1). Cotreatment (s.c.) with 1 mg/kg glutamic acid and either ultra-low-dose NTX (0.1 ng/kg) or low-dose CTX-B (0.1 mg/kg) blocked glutamic-acid-evoked hyperalgesia, and resulted in prominent analgesia (◯ and ▾, respectively), producing effects similar to those produced by ultra-low-dose NTX or CTX-B on NMDA-evoked hyperalgesia (FIG. 1). Injection of high-dose NTX (1 mg/kg) at 3 h after the initial dose of 0.1 ng/kg NTX plus 1 mg/kg glutamic acid, resulted in rapid attenuation of the initially prominent analgesia (see arrows near curves ◯ and ▾), providing further evidence that the analgesia may be mediated by opioid receptors activated by the glutamic acid-stimulated release of endogenous opioid agonists. All mice in this experiment were female (Swiss-Webster).

FIGS. 3A and 3B show that cotreatment (s.c.) of mice with ultra-low-dose NTX (0.1 ng/kg) plus monosodium glutamate (MSG) also results in prominent, long-lasting analgesia (◯). Prominent analgesia is elicited even with a lower dose of MSG (1 mg/kg) that does not evoke significant hyperalgesia when administered alone (A: ◯), as well as with a 10-fold-higher dose of MSG that evokes hyperalgesia when administered alone (B: ◯). Furthermore, delayed injection of these cotreated mice with a much higher dose of NTX blocked both hyperalgesia and analgesia (as in FIGS. 1 and 2; data not shown), indicating that the analgesia resulting from cotreatment with ultra-low-dose NTX plus MSG may be mediated by endogenous opioid mechanisms.

FIG. 4 demonstrates that cotreatment of mice with ultra-low-dose NTX blocks the acute hyperalgesic effects of a cyclic AMP-phosphodiesterase inhibitor, 3-isobutyl-1-methylxanthine (IBMX), thereby resulting in prominent analgesia. Administration of 10 mg/kg IBMX (s.c.) resulted in long-lasting hyperalgesia (>3 h)—an effect similar to that produced by NMDA, glutamic acid, or MSG (◯; cf. FIGS. 1-3). Cotreatment (s.c.) with 10 mg/kg IBMX plus ultra-low-dose NIX (0.1 ng/kg) blocked IBMX-evoked hyperalgesia, and resulted in prominent analgesia (◯). In contrast, cotreatment with 10 mg/kg IBMX plus high-dose NTX (1 mg/kg) not only blocked IBMX-evoked hyperalgesia, but also blocked onset of analgesia during the first hour after dosing (∇). During the following hour, analgesia gradually developed, probably due to the decreased level of NTX in the central nervous system, which could then selectively block excitatory opioid receptor functions (as occurs during cotreatment with 0.1 ng/kg NTX: ◯).

FIGS. 5A and 5B illustrate that cotreatment of mice with ultra-low-dose NIX blocks the acute hyperalgesic effects of a low (1 μg/kg) dose of a more specific cyclic-AMP phosphodiesterase inhibitor, rolipram, and results in prominent analgesia (A: ◯ vs. ◯). (B): A remarkably similar degree of analgesia was elicited by cotreatment with ultra-low-dose NTX plus a million-fold lower dose of rolipram (1 pg/kg) (◯). In contrast, the same ultra-low-dose of rolipram had no detectable hyperalgesic effect when administered alone (◯). Administration of a second dose of 0.1 ng/kg NTX alone to the same group of mice that were used in the experiment shown in FIG. 5A, at 24 h after the initial cotreatment, evoked significant analgesia, indicating that the single low dose of rolipram (1 μg/kg) was still effective for more than one day (data not shown).

FIGS. 6A and 6B show that cotreatment of mice with a low dose of rolipram plus CTX-B also results in prominent analgesia which can be rapidly blocked by high-dose NTX. A): Cotreatment with an extremely low dose of rolipram (1 pg/kg: ◯) plus a low dose of CTX-B (10 μg/kg) elicits prominent analgesia lasting >5 hr (◯), as occurs after cotreatment with ultra-low dose NTX (FIG. 5B). (Administration of rolipram alone does not alter baseline tail-flick latency, nor does CTX-B alone: see CTX-B data in ref. 17). B): Analgesia resulting from cotreatment with a hyperalgesic dose of rolipram (1 μg/kg; see FIG. 5A: curve ◯) plus CTX-B is rapidly blocked by delayed injection of high-dose NTX (1 mg/kg) at 3 hr after initial cotreatment (see arrow near curve ◯).

FIG. 7 illustrates that cotreatment of mice with ultra-low doses of specific mμ- or kappa-opioid receptor antagonists also block the acute hyperalgesic effects of rolipram and result in prominent analgesia. Cotreatment with the kappa opioid receptor antagonist, nor-binaltorphimine (nor-BNI, 0.1 ng/kg) results in a larger magnitude of analgesia (curve (◯) than a similar dose of the mg opioid receptor antagonist, β-funaltrexamine (β-FNA) curve (∇), although both antagonists produce analgesic effects lasting >4 hr, comparable to cotreatment with ultra-low-dose NIX (cf. FIG. 5A). Control tests with 0.1 ng/kg nor-BNI or β-FNA alone do not significantly alter baseline tail-flick latencies (data not shown).

FIGS. 8A and 8B show that cotreatment of mice with kelatorphan, an inhibitor of multiple endogenous opioid peptide-degrading enzymes (42), with an ultra-low dose of rolipram, results in rapid onset of analgesia. A): Low-dose rolipram (1 pg/kg) does not alter baseline tail-flick latency (◯), nor does administration of the enkephalinase inhibitor, kelatorphan (1 mg/kg) (◯). By contrast, cotreatment with 1 pg/kg rolipram plus 1 mg/kg kelatorphan elicits prominent analgesia (∇), even when the dose of kelatorphan is reduced to 0.1 mg/kg (∇). B): Cotreatment with 1 mg/kg kelatorphan prolongs the duration of the analgesic response to 1 pg/kg rolipram plus 0.1 ng/kg NTX (◯) vs (◯).

FIG. 9 illustrates that acute cotreatment of chronic morphine-dependent mice with ultra-low naltrexone dose blocks naloxone-precipitated withdrawal hyperalgesia and results in prominent analgesia. After chronic treatment of mice with morphine (Mor) (10 mg/kg, b.i.d. for 5 days), acute injection of 10 μg/kg naloxone (NLX) on Day 6 evokes long-lasting hyperalgesia (∇). Acute cotreatment of chronic morphine-dependent mice with an ultra-low dose of naltrexone (0.1 Ng/kg) rapidly blocks naloxone-precipitated withdrawal hyperalgesia and results in long-lasting endogenous opioid-receptor-mediated analgesia (∇). Control tests with acute injections of naloxone or naloxone+naltrexone in naive mice do not significantly alter baseline tail-flick latencies (◯ and ◯).

### DETAILED DESCRIPTION OF THE INVENTION

The present invention provides a method for treating pain in a subject, comprising administering to the subject a non-opioid agent in combination with a selective excitatory-opioid-receptor inactivator. The subject is preferably a mammal (e.g., a human; a domestic animal; or a commercial animal, including a cow, a dog, a mouse, a monkey, a pig, and a rat), and is most preferably a human.

As used herein, the term “opioid” refers to a natural or synthetic compound that binds to specific opioid receptors in the central nervous system (CNS) and peripheral nervous system (PNS) of a subject, and has agonist (activation) or antagonist (inactivation) effects at these receptors. Opioids may be endogenous (originating within the subject) or exogenous (originating outside of the subject). Opioids that have agonist (activation) effects at inhibitory opioid receptors produce analgesia. In addition, at high doses they may elicit narcosis—a non-specific and reversible depression of function of the CNS or PNS, marked by insensibility or stupor. Thus, such opioid agonists are often referred to as “narcotics,” whereas opioid antagonists (e.g., naloxone, naltrexone) are non-narcotic. Examples of opioid compounds include, without limitation, opioid alkaloids (e.g., the agonists, morphine and oxycodone, and the antagonists, naloxone and naltrexone) and opioid peptides (e.g., dynorphins, endorphins, and enkephalins). Natural opioids are “opiates,” a term which is used herein to include an opioid containing, or derived from, opium.

Opioids having agonist (activation) effects at specific opioid receptors in the CNS or PNS may be addictive. As used herein, the term “addictive” describes a substance, including an opioid, that has the potential to cause physical dependence and/or psychological dependence in a subject to whom it is administered. As further used herein, a “psychological dependence” is a psychological condition that manifests as an overpowering compulsion to continue taking an addictive substance; “physical dependence” is a state of physiologic adaptation to an addictive substance, which may increase in intensity when tolerance develops and requires increased dosage and duration of use of the addictive substance. The dependent state may manifest in an aversive withdrawal (abstinence) syndrome when the addictive substance is discontinued or its effect is counteracted by acute administration of an opioid antagonist. Additionally, as used herein, “tolerance” refers to circumstances where the dosage of an opioid agonist must be increased in order to maintain the initial analgesic effect.

The term “non-opioid” generally refers to a natural or synthetic compound that does not bind to specific opioid receptors in the nervous system, and which, therefore, does not have agonist (activation) or antagonist (inactivation) effects at these receptors. Thus, a non-opioid is neither a synthetic opioid compound nor an opiate.

A “non-opioid agent,” as used herein, is a non-opioid agent that when administered in combination with a selective excitatory-opioid-receptor inactivator, results in analgesia. Such a non-opioid agent may increase inhibitory and excitatory opioid activity in a subject to whom the non-opioid agent is administered by directly or indirectly by activating, facilitating, or stimulating one or more functions of one or more endogenous opioids in the subject (e.g., by the modulation or regulation of inhibitory and excitatory opioid receptors, particularly the activation of inhibitory and excitatory opioid receptors); by directly or indirectly causing, inducing, or stimulating the in vivo release or redistribution of one or more endogenous opioids from neurons in nociceptive networks within a subject to whom the non-opioid agent is administered; or by directly or indirectly increasing or up-regulating levels of released endogenous opioids in vivo within the subject.

Opioid-receptor activities in the subject may be enhanced by targeting endogenous opioids directly. Opioids in the subject also may be enhanced indirectly, by targeting an enzyme or other endogenous molecule that regulates or modulates the functions of endogenous opioids, or the levels of released endogenous opioids, in the subject. For example, the non-opioid agent may directly or indirectly cause the release of bimodally-acting endogenous opioid agonists that bind to and activate both inhibitory and excitatory opioid receptors. Examples of endogenous opioids that may be released in vivo within a subject, upon administration of a non-opioid agent include, without limitation, enkephalins, dynorphins, and endorphins. Furthermore, the non-opioid agent may be a hyperalgesic agent. As used herein, a “hyperalgesic agent” is an agent that has the potential to cause hyperalgesia or to enhance pain in a subject, when administered to the subject at a particular dose. “Hyperalgesia,” as further used herein, refers to excessive sensitivity or sensibility to pain.

Examples of non-opioid agents include, without limitation, excitatory amino acids (e.g., aspartic acid and glutamic acid); the salts of excitatory amino acids (e.g., N-methyl-D-aspartate (NMDA) and monosodium glutamate (MSG)); and cyclic-AMP-enhancing agents (e.g., specific cAMP phosphodiesterase (PDE) inhibitors, such as rolipram; nonspecific cAMP PDE inhibitors, including such methylxanthines as aminophylline, theophylline, 3-isobutyl-1-methylxanthine (IBMX), caffeine, and similarly-acting agents; and agents that directly enhance cAMP, such as forskolin, which stimulates synthesis of cAMP by activating adenylate cyclase). In one embodiment of the present invention, the non-opioid agent is MSG. In another embodiment of the present invention, the non-opioid agent is rolipram.

MSG has long been used throughout the world as a food-flavor enhancer, and its safety has been well documented (4, 19). MSG is also readily available, and may be easily obtained from local food stores. Accordingly, MSG presents an attractive option in the treatment of pain using non-opioid agents in combination with a selective excitatory opioid receptor inactivator.

Cyclic nucleotide PDEs are enzymes that have been grouped into seven families based upon their regulation and substrate specificity. Type IV PDEs have cAMP as their nearly-exclusive substrate. PDE inhibitors potentially increase signaling through the cAMP system by inhibiting cAMP breakdown (20). Nonspecific PDE inhibitors, such as caffeine, have long been known to improve some behavioral performance in experimental animals. Moreover, high doses of both nonspecific PDE inhibitors (e.g., IBMX) and type IV PDE-specific inhibitors (e.g., rolipram) have been found to improve memory in passive avoidance tasks in rodents when administered immediately after training. These effects may be caused by increases in cAMP concentration in the brain (20).

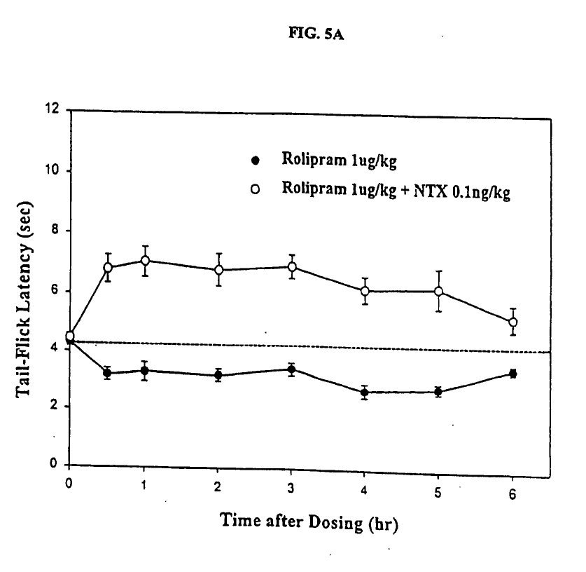

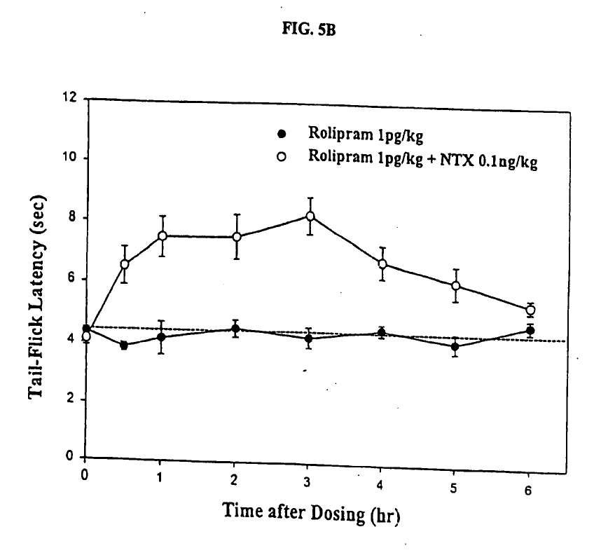

Rolipram—a type IV PDE-specific phosphodiesterase (PDE) inhibitor (20)—has been shown to increase the activity of cAMP-dependent protein kinase A (PICA), thereby affecting memory (21). Rolipram is absorbed fully and rapidly after oral administration in several species, including rat and human (20). It has been estimated that a dose of 0.1 μmol/kg of rolipram, administered subcutaneously, yields a concentration between 0.06 μM and 0.2 μM in the brain, 30 min after treatment (20). Rolipram has been clinically tested for possible enhancement of memory (21, 22) at FDA-approved doses that are a million times higher than those used herein (see FIG. 5A). Rolipram may be obtained, for example, from Research Biochemicals (Natick, Mass.) or Schering AG (Berlin, Germany). Accordingly, it is believed that rolipram, as used in accordance with the present invention, will present a safe and effective non-opioid agent.

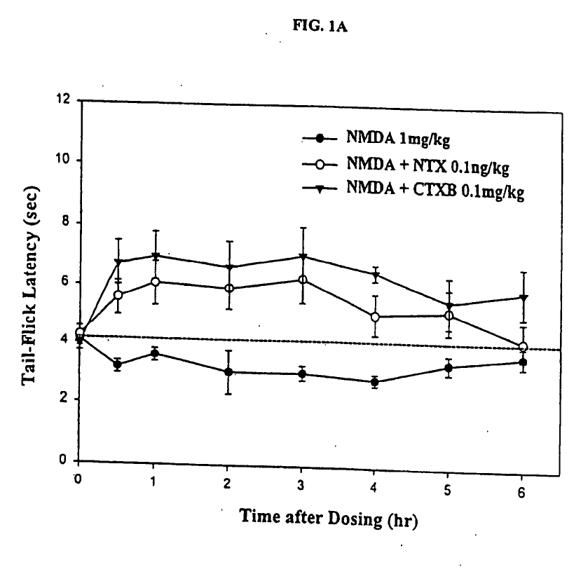

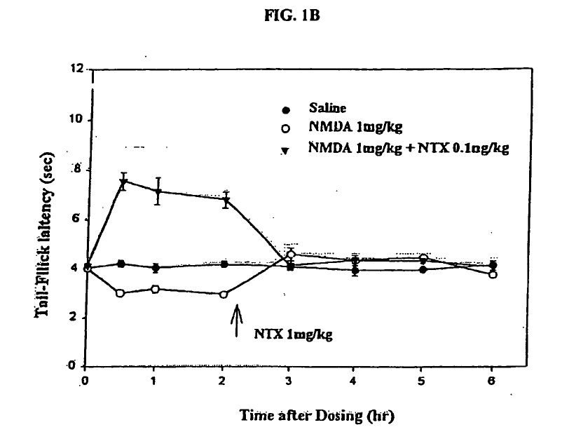

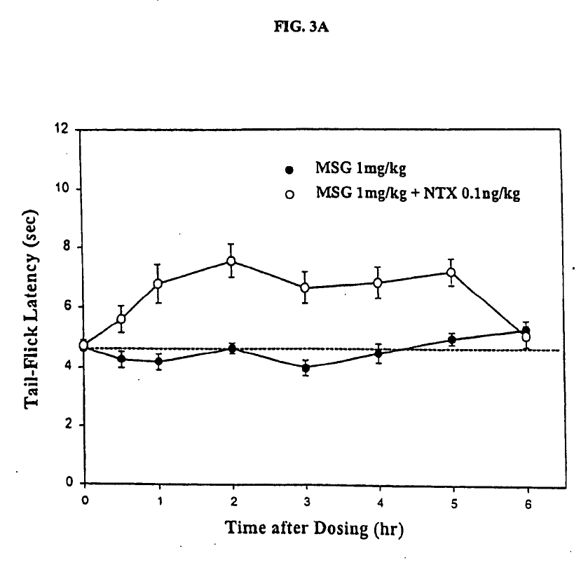

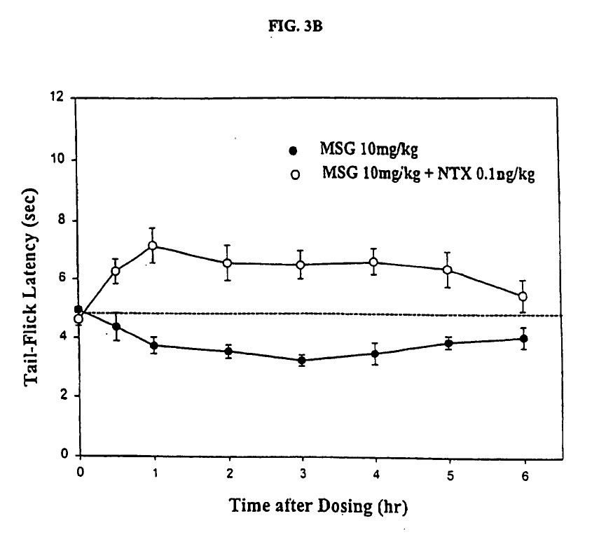

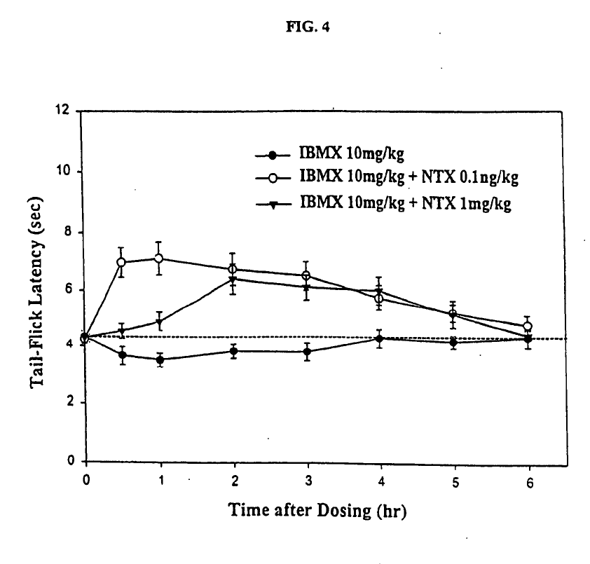

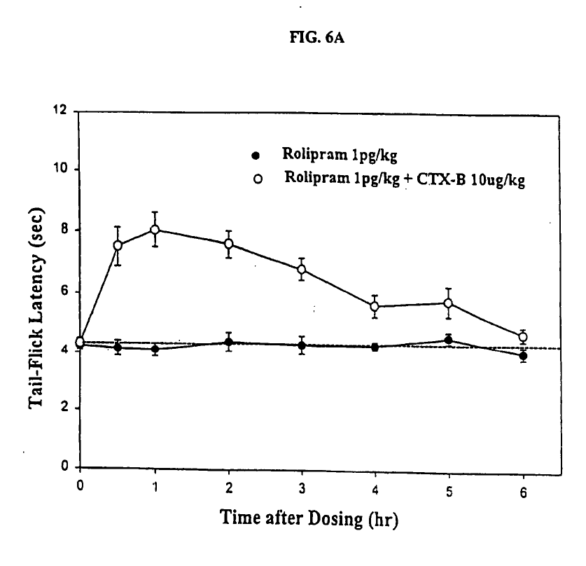

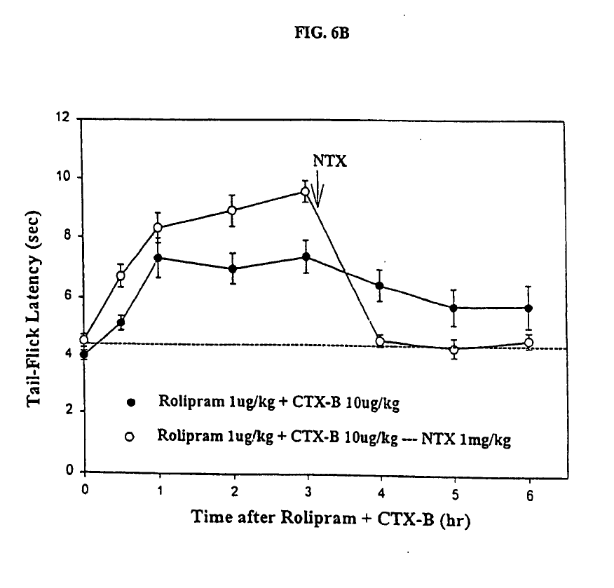

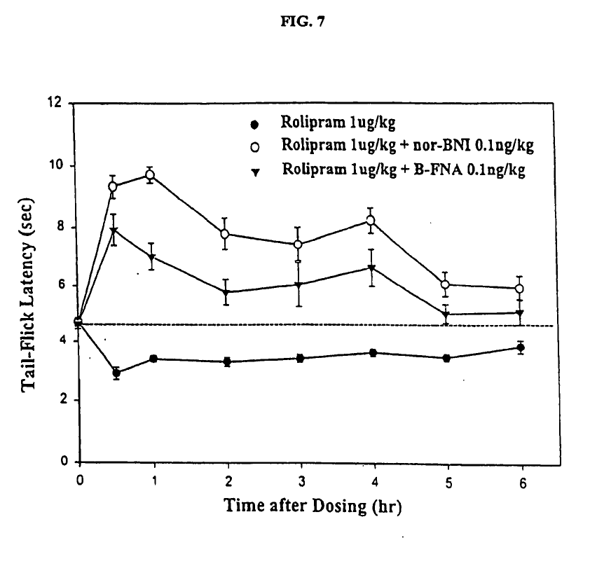

It is possible to identify, for use in the present invention, other non-opioid agents. Assays for identifying non-opioid agents that are useful in the present invention are disclosed herein (see, e.g., experimental protocols in connection with FIGS. 1-9).

The term “selective excitatory-opioid-receptor inactivator,” as used herein, refers to an agent that selectively inactivates an excitatory-opioid-receptor function. Examples of agents that selectively inactivate excitatory opioid receptor signaling include, without limitation, “selective excitatory-opioid-receptor antagonists” and “selective excitatory-opioid-receptor blockers”. The selective excitatory-opioid-receptor inactivators of the present invention also may be non-addictive. The term “non-addictive,” as used herein, refers to an opioid-receptor inactivator that does not have the potential to cause physical dependence and/or psychological dependence in a subject to whom it is administered.

As used herein, “selective excitatory-opioid-receptor antagonists” are opioid antagonists that selectively bind to, and act as antagonists to, excitatory, but not inhibitory, opioid receptors on neurons in nociceptive pathways of the nervous system. Nociceptive neurons, or nociceptors, are neurons which respond to stimuli that are damaging or potentially damaging to the skin or other body tissues (e.g., mechanical, thermal, or chemical stimuli), and which thereby mediate pain. Analgesia, or relief from pain, results from activation by opioid agonists of inhibitory opioid receptors on neurons in the nociceptive (pain) pathways of the CNS and PNS. Adverse opioid excitatory effects may result from sustained activation of excitatory opioid receptors on neurons in these nociceptive pathways. Examples of such adverse opioid excitatory effects include, without limitation, anti-analgesia, hyperexcitability, hyperalgesia, physical dependence, psychological dependence, and tolerance, as well as nausea, vomiting, diarrhea, and itching. Adverse opioid excitatory effects are attenuated by selective excitatory-opioid-receptor antagonists.

Selective excitatory-opioid-receptor antagonists suitable for use in the present invention include, without limitation, naloxone (NLX), naltrexone (NTX), nalmefene, norbinaltorphimine, diprenorphine, and similarly-acting opioid alkaloids and opioid peptides. Other selective excitatory-opioid-receptor antagonists for use in the present invention may be identified by assays such as those described for FIGS. 1-9 above. In one embodiment of the present invention, the selective excitatory-opioid-receptor inactivator is NTX. In a preferred embodiment, the non-opioid agent is rolipram, and the selective excitatory-opioid-receptor inactivator is NTX.

The term “selective excitatory-opioid-receptor blockers,” as used herein, refers to non-opioid agents that “inhibit synthesis or activity of GM1-ganglioside.” Such agents may inhibit synthesis or activity of GM1-ganglioside by directly or indirectly diminishing the levels or amount of GM1-ganglioside in a subject, or by reducing GM1-ganglioside activity in a subject by disabling, disrupting, or inactivating the functions of GM1-ganglioside in the subject, particularly the modulation or regulation of excitatory opioid receptors in nociceptive neurons. The synthesis or activity of GM1-ganglioside in a subject may be inhibited by targeting GM1-ganglioside directly. The synthesis or activity of GM1-ganglioside in a subject also may be inhibited indirectly, by targeting an enzyme or other endogenous molecule that regulates or modulates the activity or levels of GM1-ganglioside.

Examples of agents that inhibit synthesis of GM1 ganglioside include, without limitation, neuraminidase inhibitors [e.g., oseltamivir (41), zanamivir, Na₂SO₄ (42), and MgSO₄], agents that decrease or inhibit cAMP, and agents that decrease or inhibit glycosyltransferase—the enzyme that makes GM1-ganglioside. In one embodiment of the present invention, the agent that inhibits synthesis of GM1 ganglioside is a neuraminidase inhibitor.

An agent that inhibits activity of GM1-ganglioside may be, for example, an agent that is reactive with GM1-ganglioside. As used herein, “reactive” means that the agent has affinity for, binds to, or is directed against GM1-ganglioside. Such an agent may block an allosteric GM1-binding site on excitatory opioid receptors. Examples of agents that inhibit activity of GM1 ganglioside include, without limitation, the nontoxic B subunit of cholera toxin B (CTX-B), anti-GM1-ganglioside antibody, and oligonucleotide antisense to GM1-ganglioside. In one embodiment of the present invention, the agent that inhibits activity of GM1 ganglioside is cholera toxin B subunit (CTX-B).

CTX-B and its analogues and derivatives may be produced and purified from a recombinant strain of Vibrio cholerae that lacks the CTX-A gene (36). CTX-B (“choleragenoid”) and recombinant CTX-B may be obtained from List Biological Labs, Inc. (Campbell, Calif.), and can be prepared in tablet form for oral administration.

Furthermore, recombinant CTX-B (1 mg) is used in an oral cholera vaccine (“Dukoral”) produced by SBL Vaccine (Stockholm, Sweden) (35). CTX-B in the form of a spray for nasal administration also is being developed for use as a vaccine (Maxim Pharmaceuticals, San Diego, Calif.). In addition, CTX-B and CTX-B analogues may be isolated and purified from a culture of natural Vibrio cholerae using standard methods known in the art.

Neuraminidase promotes release of influenza virus from infected cells, and facilitates virus spread within the respiratory tract. Several potent and specific inhibitors of this enzyme have been developed, and two (oseltamivir and zanamivir) have been approved for human use (16). Oseltamivir is prepared in tablet form, for oral administration, under the trademark “Tamiflu”, and may be obtained from Roche Laboratories (Nutley, N.J.). Tamiflu is available as a capsule containing 75 mg of oseltamivir for oral use, in the form of oseltamivir phosphate.

Antibodies to GM1-ganglioside may be polyclonal or monoclonal, and may be produced by techniques well known to those skilled in the art. Polyclonal antibody, for example, may be produced by immunizing a mouse, rabbit, or rat with purified GM1-ganglioside. Monoclonal antibody then may be produced by removing the spleen from the immunized mouse, and fusing the spleen cells with myeloma cells to form a hybridoma which, when grown in culture, will produce a monoclonal antibody.

Other agents that inhibit activity or synthesis of GM1-ganglioside may be identified using standard in vitro assays known in the art, including binding assays. For example, a candidate agent may be contacted with nociceptive neurons in cell culture, and the level of GM1-ganglioside expression in the cells may be determined using standard techniques, such as Western blot analysis. Similarly, a candidate agent may be contacted with nociceptive neurons in cell culture, and the level of GM1-ganglioside binding activity in the cells then may be determined using a CTX-B/peroxidase assay (40). Where the level of GM1-ganglioside expression or binding activity in nociceptive neurons is reduced in the presence of the candidate, it may be concluded that the candidate could be a useful agent that inhibits GM1-ganglioside.

In the method of the present invention, administration of a non-opioid agent “in combination with” a selective excitatory-opioid-receptor inactivator refers to co-administration of the agent and the inactivator. Co-administration may occur concurrently, sequentially, or alternately. Concurrent co-administration refers to administration of both a non-opioid agent and a selective excitatory-opioid-receptor inactivator, at essentially the same time.

For concurrent co-administration, the courses of treatment with a non-opioid agent and with a selective excitatory-opioid-receptor inactivator may be run simultaneously. For example, a single, combined formulation, containing both an amount of a non-opioid agent and an amount of a selective excitatory-opioid-receptor inactivator, in physical association with one another, may be administered to the subject. The single, combined formulation may consist of an oral formulation, containing amounts of both a non-opioid agent and a selective excitatory-opioid-receptor inactivator, which may be orally administered to the subject, or a liquid mixture, containing amounts of both a non-opioid agent and a selective excitatory-opioid-receptor inactivator, which may be orally administered to the subject or injected into the subject.

It is also within the confines of the present invention that a non-opioid agent and a selective excitatory-opioid-receptor inactivator may be administered concurrently to a subject, in separate, individual formulations. For example, an amount of the non-opioid agent may be packaged in a vial or unit dose, and an amount of the inactivator may be packaged in a separate vial or unit dose, and the contents of the separate vials or unit doses then may be concurrently co-administered to the subject. Accordingly, the method of the present invention is not limited to concurrent co-administration of a non-opioid agent and a selective excitatory-opioid-receptor inactivator in physical association with one another.

In the method of the present invention, a non-opioid agent and a selective excitatory-opioid-receptor inactivator also may be co-administered to a subject in separate, individual formulations that are spaced out over a brief period of time (e.g., seconds or minutes), so as to obtain the maximum efficacy of the combination. Administration of each may range in duration, from a brief, rapid administration to a continuous perfusion. When spaced out over a brief period of time, co-administration of the non-opioid agent and the selective excitatory-opioid-receptor inactivator may be sequential or alternate.

For sequential co-administration, one of the compounds (i.e., either the agent or the inactivator) is separately administered, followed by the other within a period of seconds or minutes. For example, a full course of treatment with a non-opioid agent may be completed, and then may be immediately followed by a full course of treatment with a selective excitatory-opioid-receptor inactivator. Alternatively, for sequential co-administration, a full course of treatment with a selective excitatory-opioid-receptor inactivator may be completed, then followed by a full course of treatment with a non-opioid agent. For alternate co-administration, partial courses of treatment with a non-opioid agent may be alternated with partial courses of treatment with a selective excitatory-opioid-receptor inactivator, until a full treatment of the agent and a full treatment of the inactivator have been administered.

In the method of the present invention, a non-opioid agent and a selective excitatory-opioid-receptor inactivator are administered in amounts effective to treat pain in the subject. Pain is a complex subjective sensation, reflecting real or potential tissue damage and the affective response thereto (23). Pain may be broadly classified as acute (lasting for hours or a few days) or chronic (persisting for weeks or months), somatogenic or psychogenic. Somatogenic, or organic, pain may be explained in terms of physiologic mechanisms. Psychogenic pain occurs without an organic pathology sufficient to explain the degree of pain and disability, and is thought to be related mainly to psychologic issues (23).

Somatogenic pain may be nociceptive or neuropathic (23). Nociceptive pain results from ongoing activation of somatic or visceral pain-sensitive nerve fibers. When somatic nerves are affected, pain is typically felt as aching or pressure. Neuropathic pain results from dysfunction in the nervous system. It is believed to be sustained by aberrant somatosensory processes in the peripheral nervous system, the central nervous system, or both. Nociceptive pain may predominate in pain syndromes related to chronic joint or bone injury (e.g., arthritis, cancer, hemophilia, and sickle cell disease). Common classes of pain include acute postoperative pain, cancer pain, headaches, neuropathic pain (e.g., complex regional pain syndrome), and psychogenic pain syndromes (23). In the method of the present invention, the pain may be any of those described above, including acute pain and chronic pain, nociceptive pain and neuropathic pain.

As used herein, the phrase “effective to treat pain” means effective to ameliorate or minimize the clinical impairment or symptoms resulting from the pain (e.g., by diminishing any uncomfortable, unpleasant, or debilitating sensations experienced by the subject). The amounts of non-opioid agent and inactivator effective to treat pain in a subject will vary depending on the particular factors of each case, including the type of pain, the location of the pain, the subject's weight, the severity of the subject's condition, the agent and inactivator used, and the route of administration. A non-opioid agent and a selective excitatory-opioid-receptor inactivator may be administered to a subject in order to achieve a synergistic effect in the treatment of pain.

In one embodiment of the present invention, the amount of the non-opioid agent is an amount effective to cause hyperalgesia in a subject when administered alone. Much lower doses of the non-opioid agent also may be effective. Accordingly, in another embodiment of the present invention, the amount of the non-opioid agent is an ultra-low dose (i.e., a dose that is far below the threshold for evoking hyperalgesia in the subject when administered alone). Examples of suitable doses of the non-opioid agent include, without limitation, 1-10 mg/kg of MSG and 1 pg/kg-1 μg/kg of rolipram.

In another embodiment of the present invention, the amount of the selective excitatory-opioid-receptor inactivator is an amount effective to attenuate hyperalgesic effects associated with administration of the non-opioid agent and result in analgesia. Where the selective excitatory-opioid-receptor inactivator is a selective excitatory-opioid-receptor antagonist, the effective amount may be a low dose or an ultra-low dose of the antagonist (e.g., 1 pg/kg-1 μg/kg of NTX or NLX). Other examples of suitable doses of the selective excitatory-opioid-receptor inactivator include, without limitation, 0.01-1 mg/kg of CTX-B, 0.1-1 mg/kg of oseltamivir, and 10 mg/kg of Na₂SO₄. Oseltamivir may be administered to a subject in a dose ranging from 0.1-1 mg/kg, once or twice a day. Oseltamivir at doses that result in neuraminidase inhibition of influenza virus (16) also may be effective in decreasing GM1-ganglioside levels in a subject (41).

In accordance with the method of the present invention, the non-opioid agent and the selective excitatory-opioid-receptor inactivator (either in separate, individual formulations, or in a single, combined formulation) may be administered to a human or animal subject by known procedures, including, without limitation, nasal administration, oral administration, parenteral administration (e.g., epidural, epifascial, intracapsular, intracutaneous, intradermal, intramuscular, intraorbital, intraperitoneal (particularly in the case of localized regional therapies), intrasternal, intravascular, intravenous, parenchymatous, and subcutaneous administration), sublingual administration, transdermal administration, and administration by osmotic pump. Preferably, the non-opioid agent and the selective excitatory-opioid-receptor inactivator of the present invention are administered nasally or orally.

For nasal administration, aerosol, nasal-mist, or nasal-spray formulations of the non-opioid agent and the selective excitatory-opioid-receptor inactivator (whether individual or combined) may be prepared in accordance with standard procedures known in the art for the preparation of nasal sprays. Moreover, CTX-B in the form of a spray for nasal administration may be obtained from Maxim Pharmaceuticals (San Diego, Calif.).

For oral administration, formulations of the non-opioid agent and the selective excitatory-opioid-receptor inactivator (whether individual or combined) may be presented in solid or liquid preparations, e.g., capsules, tablets, powders, granules, dispersions, solutions, and suspensions. Such preparations are well known in the art, as are other oral-dosage forms not listed here. The formulations may have conventional additives, such as lactose, mannitol, corn starch, or potato starch. The formulations also may be presented with binders, such as crystalline cellulose, cellulose derivatives, acacia, corn starch, or gelatins. Additionally, the formulations may be presented with disintegrators, such as corn starch, potato starch, or sodium carboxymethylcellulose. The formulations also may be presented with dibasic calcium phosphate anhydrous or sodium starch glycolate. Finally, the formulations may be presented with lubricants, such as talc or magnesium stearate.

For parenteral administration, formulations of the non-opioid agent and the selective excitatory-opioid-receptor inactivator (whether individual or combined) may be combined with a sterile aqueous solution that is preferably isotonic with the blood of the subject. Such formulations may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be presented in unit or multi-dose containers, such as sealed ampules or vials. The formulations may be delivered by any mode of injection, including, without limitation, epidural, epifascial, intracapsular, intracutaneous, intradermal, intramuscular, intraorbital, intraperitoneal (particularly in the case of localized regional therapies), intrasternal, intravascular, intravenous, parenchymatous, or subcutaneous.

For transdermal administration, formulations of the non-opioid agent and the selective excitatory-opioid-receptor inactivator (whether individual or combined) may be combined with skin penetration enhancers, such as propylene glycol, polyethylene glycol, isopropanol, ethanol, oleic acid, N-methylpyrrolidone, and the like, which increase the permeability of the skin to the agent and the inactivator, and permit the agent and the inactivator to penetrate through the skin and into the bloodstream. The composition of the enhancer and the agent and/or inactivator also may be further combined with a polymeric substance, such as ethylcellulose, hydroxypropyl cellulose, ethylene/vinylacetate, polyvinyl pyrrolidone, and the like, to provide the composition in gel form, which may be dissolved in a solvent, such as methylene chloride, evaporated to the desired viscosity, and then applied to backing material to provide a patch. The agent and the inactivator may be administered transdermally, at or near the site on the subject where pain is localized. Alternatively, the agent and the inactivator may be administered transdermally at a site other than the affected area, in order to achieve systemic administration.

Formulations of the non-opioid agent and the selective excitatory-opioid-receptor inactivator (whether individual or combined) also may be released or delivered from an osmotic mini-pump or other time-release device. The release rate from an elementary osmotic mini-pump may be modulated with a microporous, fast-response gel disposed in the release orifice. An osmotic mini-pump would be useful for controlling release, or targeting delivery, of the agent and the inactivator.

For non-invasive introduction of the agent or inactivator of the present invention, micro-encapsulated preparations, such as liposomes, also may be used. Liposomal vesicles may be prepared by various methods known in the art, and liposome compositions may be prepared using any one of a variety of conventional techniques for liposome preparation known to those skilled in the art. Examples of such methods and techniques include, without limitation, chelate dialysis, extrusion (with or without freeze-thaw), French press, homogenization, microemulsification, reverse phase evaporation, simple freeze-thaw, solvent dialysis, solvent infusion, solvent vaporization, sonication, and spontaneous formation. Preparation of the liposomes may be carried out in a solution, such as an aqueous saline solution, aqueous phosphate buffer solution, or sterile water. Liposome compositions also may be prepared by various processes involving shaking or vortexing. The agent or inactivator may be incorporated into the layers of a liposome, such that its intracellular domain extends outside the liposome and its extracellular domain extends into the interior of the liposome. It is expected that liposomal delivery of a non-opioid agent or a selective excitatory-opioid-receptor inactivator will facilitate passage of the agent or inactivator through the blood-brain barrier (33).

It is also within the confines of the present invention that the formulations of the non-opioid agent and the selective excitatory-opioid-receptor inactivator (either in separate, individual formulations, or in a single, combined formulation) may be further associated with a pharmaceutically-acceptable carrier, thereby comprising a pharmaceutical composition. Accordingly, the present invention also provides a pharmaceutical composition, comprising a non-opioid agent, a selective excitatory-opioid-receptor inactivator, and a pharmaceutically-acceptable carrier. The pharmaceutical composition of the present invention would be useful for administering the non-opioid agent and the inactivator of the present invention (either in separate, individual formulations, or in a single, combined formulation) to a subject to treat pain. Where the pharmaceutical composition is administered to a subject to treat pain, the non-opioid agent and the selective excitatory-opioid-receptor inactivator are provided in amounts which are effective to treat the pain in the subject to whom the composition is administered, as described above.

The pharmaceutically-acceptable carrier of the present invention must be “acceptable” in the sense of being compatible with the other ingredients of the composition, and not deleterious to the recipient thereof. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others. It is also within the confines of the present invention to provide a separate pharmaceutical composition comprising a non-opioid agent and a pharmaceutically-acceptable carrier, and a separate pharmaceutical composition comprising a selective excitatory-opioid-receptor inactivator and a pharmaceutically-acceptable carrier.

The formulations of the pharmaceutical compositions of the present invention may be conveniently presented in unit dosage. The formulations also may be prepared by methods well-known in the pharmaceutical arts. For example, the active compound may be brought into association with a carrier or diluent, as a suspension or solution. Optionally, one or more accessory ingredients (e.g., buffers, flavoring agents, surface active agents, and the like) also may be added. The choice of carrier will depend upon the route of administration.

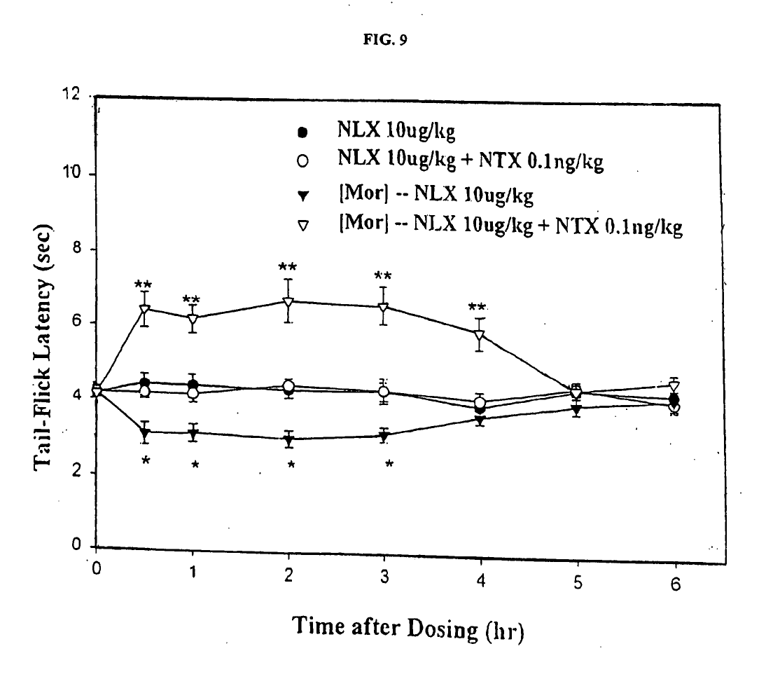

As discussed above, previous patents have disclosed that ultra-low doses of naltrexone, alone or in combination with low-dose methadone (e.g., Re. 36,547), and ultra-low doses of other excitatory-opioid-receptor antagonists alone (e.g., U.S. Pat. Nos. 5,580,876 and 5,767,125), can provide effective, long-term maintenance treatment for opioid addiction after acute detoxification, and can prevent relapse to drug abuse. As discussed in the description to FIG. 9, it has been shown that NLX-precipitated withdrawal hyperalgesia in chronic morphine-dependent mice can be rapidly converted to analgesia by cotreatment with ultra-low dose NTX. This provides strong evidence that the method of cotreatment of the present invention will provide an alternate, effective means of diminishing an opioid-addict's dependence on exogenous opioid, by boosting his/her endogenous opioids, and by rapidly converting aversive hyperalgesic withdrawal effects to analgesic effects.

The present invention further provides a method for treating opioid-withdrawal effects in a subject, comprising administering to the subject a non-opioid agent in combination with a selective excitatory-opioid-receptor inactivator, in amounts effective to treat opioid-withdrawal effects in the subject. The withdrawal effects may be acute or protracted. The subject is preferably a mammal (e.g., a human; a domestic animal; or a commercial animal, including a cow, a dog, a mouse, a monkey, a pig, and a rat), and is more preferably a human. Even more preferably, the subject is an opioid addict, particularly a detoxified opioid addict.

The term “treating opioid-withdrawal effects,” as used herein, means treating any one or more of the effects produced in an opioid-addicted subject as a result of withdrawal from the opioid. Adverse side-effects resulting from opioid withdrawal may remain in a subject for a protracted period of time, even after acute withdrawal effects have subsided. Indeed, it has been demonstrated in vitro that chronic morphine-treated sensory ganglion neurons can remain supersensitive to the excitatory effects of NLX for months after the culture medium has returned to normal (3). Examples of protracted opioid-withdrawal effects that may be treated by the method of the present invention include, without limitation, anti-analgesia, hyperexcitability, hyperalgesia, physical dependence, psychological dependence, and tolerance, as well as nausea, vomiting, diarrhea, and itching. In the method of the present invention, the non-opioid agent and the excitatory-opioid-receptor activator, their formulations in pharmaceutical compositions, and there mode of administration are as described above.

The method of the present invention permits chronic treatment of protracted opioid-withdrawal effects in a subject, particularly a detoxified opioid-addicted subject, through the long-term administration of a non-opioid agent in combination with a selective excitatory-opioid-receptor inactivator. As used herein, “long-term administration” means administration for at least three weeks.

In the method of the present invention, the non-opioid agent and the selective excitatory-opioid-receptor inactivator are administered in amounts that are effective to treat opioid-withdrawal effects in the subject to whom the composition is administered. As used herein, the phrase “effective to treat opioid-withdrawal effects” means effective to ameliorate, attenuate, minimize, or terminate the acute or protracted clinical impairment or long-term symptoms resulting from opioid withdrawal, including anti-analgesia, hyperexcitability, hyperalgesia, physical dependence, psychological dependence, and tolerance, as well as nausea, vomiting, diarrhea, and itching. The amounts of non-opioid agent and inactivator effective to treat opioid-withdrawal effects in a subject will vary depending on the particular factors of each case, including the types of protracted opioid-withdrawal effects, the severity of the effects, the subject's weight, the agent and inactivator used, and the route of administration. A non-opioid agent and a selective excitatory-opioid-receptor inactivator may be administered to a subject in order to achieve a synergistic effect in the treatment of opioid-withdrawal effects.

In one embodiment of the present invention, the amount of the non-opioid agent is an amount effective to cause hyperalgesia in a subject when administered alone. Much lower doses of the non-opioid agent also may be effective. Accordingly, in another embodiment of the present invention, the amount of the non-opioid agent is an ultra-low dose (i.e., a dose that is far below the threshold for evoking hyperalgesia in the subject when administered alone). Examples of suitable doses of the non-opioid agent include, without limitation, 1-10 mg/kg of MSG and 1 pg/kg-1 μg/kg of rolipram.

In another embodiment of the present invention, the amount of the selective excitatory-opioid-receptor inactivator is an amount effective to attenuate hyperalgesic effects associated with administration of the non-opioid agent and result in analgesia. Where the selective excitatory-opioid-receptor inactivator is a selective excitatory-opioid-receptor antagonist, the effective amount may be an ultra-low dose of the antagonist (e.g., 1 pg/kg-1 μg/kg of NTX or NLX). Other examples of suitable doses of the selective excitatory-opioid-receptor inactivator include, without limitation, 0.01-1 mg/kg of CTX-B, 0.1-1 mg/kg of oseltamivir, and 10 mg/kg of Na₂SO₄. Oseltamivir may be administered to a subject in a dose ranging from 0.1-1 mg/kg, once or twice a day. Oseltamivir at doses that result in neuraminidase inhibition of influenza virus (16) also may be effective in decreasing GM1-ganglioside levels in a subject (41).

Lastly, the present invention provides a method for treating opioid-withdrawal effects in a subject, comprising administering to the subject a non-narcotic agent in combination with a selective excitatory-opioid-receptor inactivator, in amounts effective to treat opioid-withdrawal effects in the subject. The non-narcotic agent is preferably a dose of naloxone sufficient to precipitate withdrawal hyperalgesia if administered alone and the excitatory opioid receptor-inactivator is preferably ultra-low dose naltrexone (see FIG. 9).

The present invention is described in the following Experimental Details section, which is set forth to aid in the understanding of the invention, and should not be construed to limit in any way the scope of the invention as defined in the claims which follow thereafter.

### Experimental Details

The present invention is based in part upon the discovery that hyperalgesia evoked in normal, naïve mice, for periods lasting >4-5 h, by acute administration of a relatively low dose of glutamate, N-methyl-D-aspartate (NMDA), 3-isobutyl-1-methylxanthine (IBMX), or rolipram, can be rapidly blocked and reversed to prominent, long-lasting analgesia by cotreatment with ultra-low-dose NTX (FIGS. 1-5). Furthermore, much lower doses of some of these non-opioid agents are effective even at doses that are far below the threshold for evoking hyperalgesic effects. For example, as disclosed herein, 1 pg/kg rolipram can elicit prominent, long-lasting analgesia in normal mice when cotreated with ultra-low-dose NTX, or with CTX-B or oseltamivir (FIG. 5B). Other animal studies suggest that related excitatory amino acids (e.g., aspartate) and related cyclic-AMP-enhancing agents (e.g., caffeine) may evoke similar analgesic effects when cotreated with ultra-low-dose NTX.

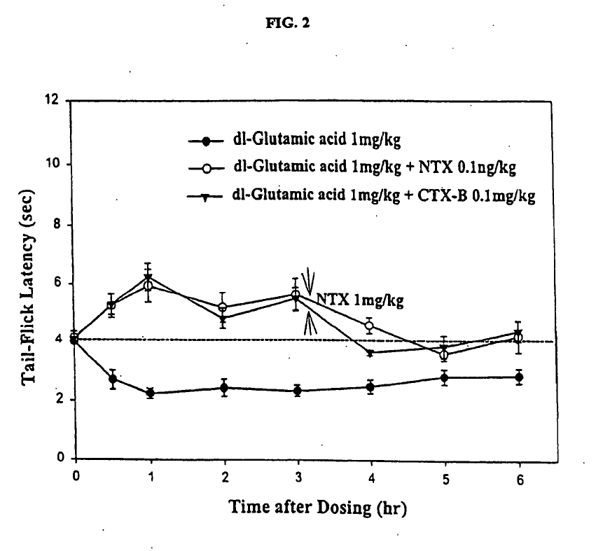

The analgesia resulting from the non-narcotic cotreatment procedure described herein is mediated by activation of opioid receptors, because it can be antagonized rapidly by injection of a high dose of NTX (1 mg/kg), at 2-3 h after initial cotreatment (FIGS. 1B and 2). The results suggest that the hyperalgesia evoked by glutamate, NMDA, IBMX, or rolipram may be due to the release of endogenous opioid peptides, in nociceptive pathways, at relatively low concentrations. Such release would be expected to stimulate both excitatory and inhibitory opioid receptors, but low quantities of endogenous opioid agonists will preferentially activate excitatory opioid hyperalgesic effects [as has been shown to occur during administration to naïve mice of very low μg/kg doses of exogenous morphine (29)]. Furthermore, combining ultra-low-dose NTX with MSG should markedly attenuate hyperalgesic effects (e.g., headache and other adverse reactions) that are evoked by dietary quantities of MSG in some people.

It was unexpected that cotreatment with glutamate (or ultra-low-dose rolipram) plus ultra-low-dose NTX would evoke long-lasting analgesia mediated by endogenous opioids (e.g., enkephalins, dynorphins), in view of the well-known metabolic instability of these peptides, which are rapidly degraded by enkephalinases (e.g., aminopeptidase) (14, 15). Extensive research projects have been carried out during the past two decades to develop various inhibitors of endogenous opioid-peptide-degrading enzymes that would attenuate the rapid breakdown of endogenous opioid peptides, and thereby enhance endogenous opioid analgesia (11, 12, 14, 15).

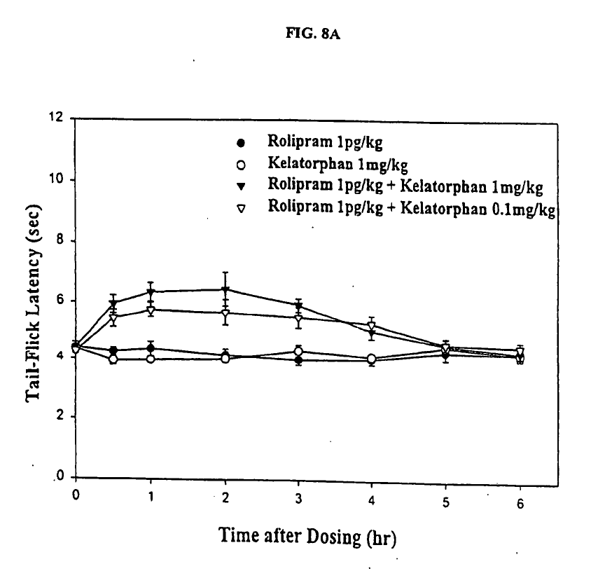

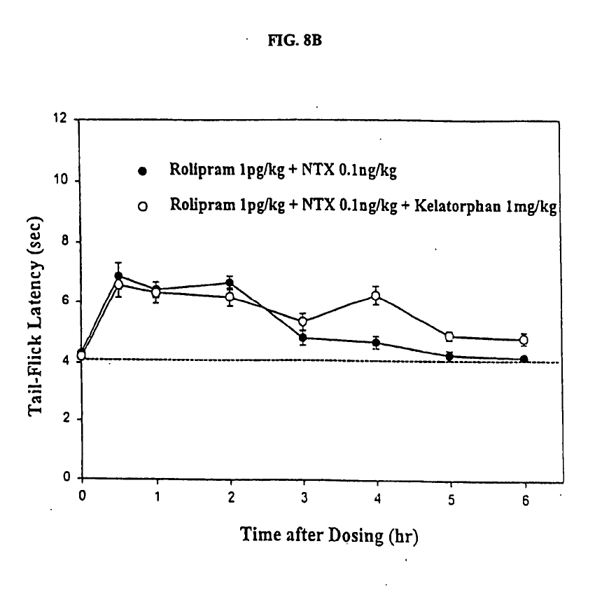

In contrast, cotreatment with glutamate, or low-dose rolipram, plus ultra-low-dose NTX demonstrates that the metabolic instability of endogenous opioids does not preclude generation of significant analgesic efficacy, as long as their excitatory side-effects are selectively blocked by ultra-low-dose NTX. Furthermore, the present invention predicts that cotreatment of pain patients with an inhibitor of endogenous opioid-peptide-degrading enzymes (15) plus ultra-low-dose NTX will markedly increase the analgesic potency of endogenous opioids following appropriate enkephalinase-inhibitor treatment (FIG. 8). Thus, selective blockade, by ultra-low-dose NTX of tonic excitatory but not inhibitory opioid-receptor-mediated activity, induced by release of endogenous bimodally-acting opioid agonists, provides a simple mechanism that may underlie the efficacy of the proposed cotreatment with excitatory amino acids or cyclic AMP enhancers plus ultra-low-dose NTX.

It should be emphasized that cotreatment with an agent plus ultra-low-dose NTX results in significant endogenous opioid receptor-mediated analgesia, without the use of exogenous morphine (or any other narcotic opioid agonist). This effect eliminates the complex adverse CNS side-effects that have heretofore required DEA restrictions on clinical administration to pain patients of conventional opioid analgesics. Therefore, the novel cotreatment preparation described herein provides a remarkably simple method to treat pain and to enhance the pharmacological analgesic properties of endogenous bimodally-acting opioid agonists with extremely low risk of aversive side-effects, even in comparison with common “over-the-counter” analgesics, e.g., aspirin and acetaminophen.

The preferred non-opioid component for cotreatment with ultra-low-dose NTX is MSG, in view of its worldwide, long-term use as a safe food-flavor enhancer. The well-documented safety of MSG (4, 19) and of ultra-low-dose NTX may even permit use of novel combination tablets with fewer adverse side-effects than conventional, over-the-counter non-narcotic and non-addictive analgesics (e.g., aspirin and acetaminophen).

Another preferred non-opioid component for cotreatment with ultra-low-dose NTX is ultra-low-dose rolipram. Previously, it was shown that administration of moderately low doses of this specific cyclic AMP phosphodiesterase inhibitor (˜1 μg/kg) in rodents and humans resulted in significant enhancement in memory behavior, with relatively moderate adverse side-effects (20, 21, 22). It has been shown herein that a thousand- to a million-fold lower dose of rolipram in mice, when co-administered together with ultra-low-dose NTX, can elicit a remarkable degree of opioid receptor-mediated, long-lasting analgesia. This cotreatment may provide an even superior non-narcotic and non-addictive analgesic preparation.

### REFERENCES

1. Buchsbaum et al., Naloxone alters pain perception and somatosensory evoked potentials in normal subjects. Nature, 270:620-22, 1977.
2. Cappell et al., Enhancement of naloxone-induced analgesia by pretreatment with morphine. Pharmacol. Biochem. Behavior, 34:425-27, 1989.
3. Crain and Shen, Chronic morphine-treated sensory ganglion neurons remain supersensitive to the excitatory effects of naloxone for months after return to normal culture medium: an in vitro model of ‘protracted opioid dependence’. Brain Res., 694(1-2):103-10, Oct. 2, 1995.
4. Geha et al., Review of alleged reaction to monosodium glutamate and outcome of a multicenter double-blind placebo-controlled study. J. Nutr., 130:1058S-62S, 2000.
5. Gillman and Lichtigfeld, Naloxone analgesia: an update. Intern. J. Neurosci., 48:321-24, 1989.
6. Greeley et al., “Paradoxical” analgesia induced by naloxone and naltrexone. Psychopharmacol., 96:36-39, 1988.
7. Kayser and Guilbaud, Dose-dependent analgesic and hyperalgesic effects of systemic naloxone in arthritic rats. Brain Res., 226:344-48, 1981.
8. Kayser et al., Analgesia produced by low doses of the opiate antagonist naloxone in arthritic rats is reduced in morphine-tolerant animals. Brain Res., 371:37-41, 1986.
9. Lasagna, L., Drug interaction in the field of analgesic drugs. Proc. Roy. Soc. Med., 58:978-83, 1965.
10. Levine et al., Naloxone dose-dependently produces analgesia and hyperalgesia in postoperative pain. Nature, 278:740-41, 1979.
11. Noble et al., Inhibition of the enkephalin-metabolizing enzymes by the first systemically active mixed inhibitor prodrug RB101 induces potent analgesic responses in mice and rats. J. Pharmacol. Exp. Ther., 261:181-90, 1992.
12. Noble et al., Pain-suppressive effects on various nociceptive stimuli (thermal, chemical, electrical and inflammatory) of the first orally active enkephalin-metabolizing enzyme inhibitor RB 120. Pain, 73:383-91, 1997.
13. Poulos et al., Naloxone-induced analgesia and morphine supersensitivity effects are contingent upon prior exposure to analgesic testing. Psychopharmacol., 100:31-35, 1990.
14. Rogues et al., The enkephalinase inhibitor thiorphan shown antinociceptive activities in mice. Nature, 288:286-88, 1980.
15. Rogues, B. P., Novel approaches to targeting neuropeptide systems. Trends Pharmacol. Sci., 21:475-483, 2000.
16. Gubareva et al., Influenza virus neuraminidase inhibitors, Lancet, 355:827-35, 2000.
17. Shen and Crain, Cholera toxin-B subunit blocks excitatory opioid receptor-mediated hyperalgesic effects in mice, thereby unmasking potent opioid analgesia and attenuating opioid tolerance/dependence. Brain Res., 919(1):20-30, Nov. 16, 2001.
18. Walker et al., Chronic selective blockade of opioid receptors produces analgesia and augmentation of the effects of a k agonist. Brain Res., 538:181-86, 1991.
19. Walker and Lupien, The safety evaluation of monosodium glutamate. J. Nutr., 130:1049S-52S, 2000.
20. Barad et al., Rolipram, a type IV-specific phosphodiesterase inhibitor, facilitates the establishment of long-lasting long-term potentiation and improves memory. Proc. Natl. Acad. Sci. USA, 95(25):15020-25, Dec. 8, 1998.
21. Cohen, P., It's all in the mind. Keystone Millennium Meeting: Conference Report, 12-15, September 2000.
22. Bach et al., Age-related defects in spatial memory are correlated with defects in the late phase of hippocampal long-term potentiation in vitro and are attenuated by drugs that enhance the cAMP signaling pathway. Proc. Natl. Acad. Sci. USA, 96(9):5280-85, Apr. 27, 1999.
23. Beers and Berkow (eds.), The Merck Manual of Diagnosis and Therapy, 17^{th} ed. (Whitehouse Station, N.J.: Merck Research Laboratories, 1999) 1363, 1369-1375, 1585.
24. Crain and Shen, Opioids can evoke direct receptor-mediated excitatory effects on sensory neurons. Trends in Pharmacol. Sci., 11:77-81, 1990.
25. Crain and Shen, Ultra-low concentrations of naloxone selectively antagonize excitatory effects of morphine on sensory neurons, thereby increasing its antinociceptive potency and attenuating tolerance/dependence during chronic cotreatment. Proc. Natl. Acad. Sci. USA, 92:10,540-44, 1995.
26. Crain and Shen, GM1 ganglioside-induced modulation of opioid receptor-mediated functions. Ann. N.Y. Acad. Sci., 845:106-25, 1998.
27. Crain and Shen, Modulation of opioid analgesia, tolerance and dependence by Gₛ-coupled, GM1 ganglioside-regulated opioid receptor functions. Trends in Pharmacol. Sci., 19:358-65, 1998.
28. Crain and Shen, Antagonists of excitatory opioid receptor functions enhance morphine's analgesic potency and attenuate opioid tolerance/dependence liability. Pain, 84:121-31, 2000.
29. Crain and Shen, Acute thermal hyperalgesia elicited by low-dose morphine in normal mice is blocked by ultra-low naltrexone, unmasking potent opioid analgesia. Brain Res., 888:75-82, 2001.
30. Fishman, P. H., Role of membrane gangliosides in the binding and action of bacterial toxins. J. Membr. Biol., 69:85-97, 1982.
31. Fishman, P. H., Recent advances in identifying the functions of gangliosides. Chem. Phys. Lipids., 42:137-51, 1986.
32. Fishman, P. H., Gangliosides and cell surface receptors and transducers of biological signals. In New Trends in Ganglioside Research: Neurochemical and Neuroregenerative Aspects, R. W. Ledeen, E. L. Hogan, G. Tettamenti, A. J. Yates, and R. K. Yu (eds.) (Padova: Liviana, 1988) 183-201.
33. Harokopakis et al., Effectiveness of liposomes possessing surface-linked recombinant B subunit of cholera toxin as an oral antigen delivery system. Infect. and Immun., 66:4299-304, 1998.
34. Ledeen, R. W., Biosynthesis, metabolism, and biological effects of gangliosides. In Neurobiology of Glycoconjugates, R. V. Margolis and R. K. Margolis (eds.) (New York: Plenum, 1989) 43-83.
35. Rudin et al., Differential kinetics and distribution of antibodies in serum and nasal and vaginal secretions after nasal and oral vaccination of humans. Infect. Immun., 66:3390-96, 1998.
36. Sanchez and Holmgren, Recombinant system for overexpression of cholera toxin B subunit in Vibrio cholerae as a basis for vaccine development. Proc. Natl. Acad. Sci. U.S.A., 86:481-85, 1989.
37. Shen and Crain, Dual opioid modulation of the action potential duration of mouse dorsal root ganglion neurons in culture. Brain Res., 491:227-42, 1989.
38. Shen and Crain, Chronic selective activation of excitatory opioid receptor functions in sensory neurons results in opioid “dependence” without tolerance. Brain Res., 597:74-83, 1992.
39. Shen and Crain, Ultra-low doses of naltrexone or etorphine increase morphine's antinociceptive potency and attenuate tolerance/dependence in mice. Brain Res., 757:176-90, 1997.
40. Wu et al., The role of GM1 ganglioside in regulating excitatory opioid effects. Ann. N.Y. Acad. Sci., 845:126-38, 1998.
41. Crain, S. M. and Shen, K.-F., Neuraminidase inhibitor, oseltamivir blocks GM1 ganlioside-regulated excitatory opioid receptor-mediated hyperalgesia, enhances opioid analgesia and attenuates tolerance in mice, Brain Res., 995: 260-266, 2004.
42. Greffard, A., et al., Inhibition of acid sialidase by inorganic sulfate, Biochimica et Biophysica Acta, 1334: 140-148, 1997.

All publications mentioned hereinabove are hereby incorporated in their entireties. While the foregoing invention has been described in some detail for purposes of clarity and understanding, it will be appreciated by one skilled in the art, from a reading of the disclosure, that various changes in form and detail can be made without departing from the true scope of the invention in the appended claims.

1. A method for treating pain in a subject in need of treatment comprising administering to the subject a non-opioid agent in combination with a selective excitatory-opioid-receptor inactivator, in amounts effective to treat pain in the subject.

2. A method for treating opioid-withdrawal effects in a subject comprising administering to the subject of a non-opioid agent in combination with a selective excitatory-opioid-receptor inactivator, in amounts effective to treat opioid-withdrawal effects in the subject.

3. The method of claim 1, wherein the non-opioid agent and the selective excitatory-opioid-receptor inactivator induce analgesia.

4. The method of claim 1, wherein the non-opioid agent is an excitatory amino acid, a salt of an excitatory amino acid, or a cyclic-AMP (cAMP) enhancer.

5. The method of claim 4, wherein the excitatory amino acid is aspartic acid or glutamic acid.

6. The method of claim 4, wherein the salt of an excitatory amino acid is N-methyl-D-aspartate (NMDA) or monosodium glutamate (MSG).

7. The method of claim 1, wherein the non-opioid agent is MSG.

8. The method of claim 4, wherein the cAMP enhancer is a cAMP phosphodiesterase (PDE) inhibitor or an agent that directly enhances cAMP.

9. (canceled)

10. The method of claim 8, wherein the cAMP PDE inhibitor is rolipram.

11. The method of claim 8, wherein the cAMP PDE inhibitor is a methylxanthine.

12. The method of claim 11, wherein the methylxanthine is aminophylline, theophylline, 3-isobutyl-1-methylxanthine (IBMX), or caffeine.

13. The method of claim 8, wherein the agent that directly enhances cAMP is forskolin.

14. The method of claim 1, wherein the non-opioid agent is rolipram.

15. The method of claim 1, wherein the selective excitatory-opioid-receptor inactivator is a selective excitatory-opioid-receptor antagonist or a selective excitatory-opioid-receptor blocker.

16. The method of claim 15, wherein the selective excitatory-opioid-receptor antagonist is diprenorphine, naloxone, naltrexone (NTX), nalmefene or norbinaltorphimine.

17. The method of claim 1, wherein the selective excitatory-opioid-receptor inactivator is NTX.

18. The method of claim 1, wherein the non-opioid agent is rolipram, and the selective excitatory-opioid-receptor inactivator is NTX.

19. The method of claim 15, wherein the selective excitatory-opioid-receptor blocker is an agent that inhibits synthesis or activity of GM1 ganglioside.

20. The method of claim 19, wherein the agent that inhibits synthesis of GM1 ganglioside is a neuraminidase inhibitor.

21. The method of claim 20, wherein the neuraminidase inhibitor is oseltamivir, zanamivir, MgSO₄, or Na₂SO₄.

22. The method of claim 19, wherein the agent that inhibits activity of GM1 ganglioside is cholera toxin B subunit (CTX-B).

23. The method of claim 1, wherein the amount of the non-opioid agent is an amount effective to cause hyperalgesia in the subject when administered alone.

24. The method of claim 23, wherein the amount of the non-opioid agent is a low dose that does not elicit hyperalgesia.

25. The method of claim 1, wherein the amount of the selective excitatory-opioid-receptor inactivator is an amount effective to attenuate hyperalgesic effects.

26-27. (canceled)

28. The method of claim 1, wherein the mode of administration is selected from the group consisting of nasal, oral, parenteral, sublingual, and transdermal.

29. The method of claim 28, wherein the mode of administration is nasal or oral.

30. A pharmaceutical composition comprising a non-opioid agent and a selective excitatory-opioid-receptor inactivator, and a pharmaceutically-acceptable carrier.

31-50. (canceled)

51. A method for treating opioid-withdrawal effects in a subject, comprising administering to the subject a non-narcotic agent in combination with a selective excitatory-opioid-receptor inactivator, in amounts effective to treat opioid-withdrawal effects in the subject.

52. (canceled)
